(19) United States
(12) Patent Application Publication
(10) Pub. No.: US 2010/0255465 A1
(43) Pub. Date: Oct. 7, 2010

(54) Nucleic Acid-Polymer Particle for and Method of Tracing Movement of a Liquid

(75) Inventor: Robert Sleat, Cardiff (GB)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: EnviroGene Ltd.

(21) Appl. No.: 12/308,596

(22) PCT Filed: Jun. 18, 2007
(86) PCT No.: PCT/GB2007/002254

§ 371 Date: Jun. 8, 2010

(30) Foreign Application Priority Data
Jun. 19, 2006 (GB) 0612130.5

(51) Int. Cl. C12Q 1/68; C07H 21/04; C08G 63/44

(52) U.S. Cl. 435/6; 536/23.1; 525/418

(57) ABSTRACT

A particle (1) for tagging a liquid. The particle comprises a nucleic acid tag; a carrier nucleic acid; and a linear polymer. The linear polymer has a backbone comprising amido and tertiary amino groups arranged regularly on the backbone. The nucleic acid tag and the carrier nucleic acid have different sequences. The particle comprises at least ten times more carrier nucleic acid than nucleic acid tag. Use of particles for marking materials in which the particles comprise the said linear polymer and a nucleic acid.

The present invention relates to a method of marking a material (e.g. a liquid) and, in particular, a method of marking a material using a nucleic acid. The invention also relates to a particle or bead for tagging a material, the particle or bead comprising a nucleic acid tag.

There are many circumstances in which it is useful to track the movement of a material in the environment. One category of such circumstances are so-called “connectivity studies”, for example, to determine the points of leakage of a water main. Another example is to determine the time of transit of drainage water or in water catchment area studies. A more specific example is in the field of pollution studies. For instance, a local authority may become aware that a beach is being contaminated with animal excrement which has been washed down from neighbouring fields and farms. In order to determine the precise source or sources of the contamination, it is desirable to track the passage of material from suspected source areas in the farms and fields and determine whether materials from the suspected sources do, in fact, arrive at the beach in question. In response to such a study, it is possible to clean up and prevent animal access to the actual sources of pollution.

It is known to use fluorescent tags to mark materials in such circumstances. However, there are problems with this approach, primarily because of a limit to the number of readily distinguishable tags in existence. In view of the limited number of tags available, often only one study can be carried out in a particular area at a time. Furthermore, if a study is to be repeated, sufficient time must be allowed for the original tag to disperse in the environment so as to avoid any tag from the original study being counted in the repeated study.

There have previously been proposals for the marking of materials using nucleic acid tags. For example, WO91/17265 discloses the monitoring of the movement of a material by adding DNA molecules and then sampling the resulting material, after movement thereof, and detecting the presence of the DNA molecules in the sample.

WO95/02702 discloses the marking of a solid material comprising applying to the material “microbeads” comprising a nucleic acid tag.

WO00/61799 discloses the marking of a material with a nucleic acid tag, which is applied directly to the material. The quantity of the nucleic acid tag present in a sample of the material is then determined to provide an indication of the quantity of the marker present in the material.

In all of these prior proposals, the nucleic acid tags are either added “naked” to the material to be marked or are covalently bound to the surface of a bead or similar particle which is applied to the material. While such procedures are acceptable to mark protected materials, such as manufactured goods, the problem of marking materials in the environment in such a way is that the nucleic acid tags tend to be degraded over time. For example, the tags may be degraded by enzymatic activity and possibly by ultraviolet light. Furthermore, in the case of nucleic acid tags attached to the surface of beads, crosslinking may occur between the nucleic acid tags resulting in clumping together of the tags which can make detection of the tag difficult or even impossible.

There have also been proposals to encapsulate nucleic acid tags within a bead. For example, it has been proposed to encapsulate nucleic acid tags in polypropylene beads. The problem with this approach is that the process of extruding the polypropylene beads requires high temperatures and, in practice, a small quantity of residual water remains in the polypropylene material. The effect of the high temperature and the water on the nucleic acids being capsulated is to cause hydrolysis of the nucleic acid tag rendering it useless. Another problem with such an approach is that, after a material has been sampled, it is technically difficult to extract the nucleic acid tags from the polypropylene beads in order to determine the sequence of the tag.

The present invention seeks to alleviate one or more of the above problems.

According to one aspect of the present invention, there is provided a particle for tagging a material, the particle comprising:
a nucleic acid tag;
carrier nucleic acid; and
a linear polymer with a backbone comprising amido and tertiary amino groups arranged regularly on the backbone.

Advantageously, the nucleic acid tag and the carrier nucleic acid have different sequences.

Preferably, the particle comprises at least ten times more carrier nucleic acid than nucleic acid tag.

Conveniently, the particle has a size in the range of 130 to 200 nm.

Preferably the nucleic acid tag is between 80 and 100 bp long.

Advantageously, the nucleic acid tag is single stranded DNA.

Conveniently, the particle comprises a molar ration of between 1:1000 and 1:10000 of nucleic acid tag to carrier nucleic acid tag.

Preferably, the carrier nucleic acid tag comprises a naturally occurring sequence more preferably salmon testes DNA, or maize DNA.

According to another aspect of the present invention, there is provided a method of marking a material comprising the steps of:
i) providing one or more marker particles; and
ii) applying the marker particles to the material, wherein the marker particles comprise a linear polymer with a backbone comprising amido and tertiary amino groups arranged regularly on the backbone and a nucleic acid tag.

Conveniently, the nucleic acid tag is from 80 to 100 bp long.

Preferably each marker particle further comprises carrier nucleic acid having a different sequence from the nucleic acid tag.

Advantageously, each marker particle comprises a molar ratio of between 1:1000 and 1:10000 of nucleic acid tag to carrier nucleic acid tag.

Conveniently, the carrier nucleic acid comprises a naturally occurring sequence, preferably salmon testes DNA, or maize DNA.

Preferably, the method further comprises the step of, after step ii), exposing the material to the environment. That is to say, the movement of the material is allowed to proceed unchecked. In some embodiments, this occurs through entirely natural processes (e.g. the material is washed down a river) but in other embodiments some human intervention occurs (e.g. movement of material through sewer pipes) although the precise destination of the material remains uncertain.

In accordance with a further aspect of the present invention, there is provided a method of detecting whether a material has been marked as described above comprising the steps of:
i) sampling a portion of the material; and
ii) detecting the presence of the nucleic acid tag in the sample

Conveniently, step ii) further comprises the step of concentrating the amount of nucleic acid tag in the sample by filtration.

Preferably, step ii) further comprises the step of extracting the nucleic acid tag from the marker particles.

Advantageously, step ii) further comprises the step of determining the quantity of nucleic acid tag present in the sample.

Conveniently, the quantity of nucleic acid tag present in the sample is determined by real time PCR.

Preferably, each material is marked with a separate set of marker particles, the marker particles in each set comprising nucleic acid tag having a different sequence.

Advantageously, the nucleic acid tags in each set of marker particles comprise a different identifying sequence flanked by generic sequences shared by the nucleic acid tags in all sets.

According to yet another aspect of the invention there is provided the use of a composition comprising a linear polymer with a backbone comprising amido and tertiary amino groups arranged regularly on the backbone and a nucleic acid tag, for marking a material.

Conveniently, the linear polymer comprises or consists of a poly(amidoamine).

Preferably, the linear polymer further comprises ethylene glycol or poly(ethylene glycol).

Advantageously, said linear polymer is a poly(ethylene glycol)-poly(amidoamine) block copolymer or ethylene glycol-poly(amidoamine) block copolymer.

Conveniently, said linear polymer is a block copolymer with the structure
[poly(amidoamine)-(ethylene glycol)_{y}]ₓ
   wherein x is from 1 to 50 and each y is independently from 1 to 200 whenever it occurs.

Alternatively, said linear polymer is a block copolymer with the structure
(ethylene glycol)_{y}-poly(amidoamine)-ethylene glycol)_{y}
   wherein each y is independently 1 to 200.

Preferably, said poly(amidoamine) has the formula:

(a)

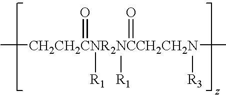

or (b)

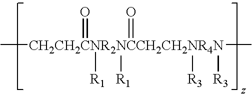

or (c)

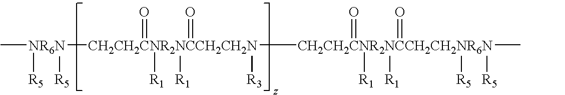

or (d)

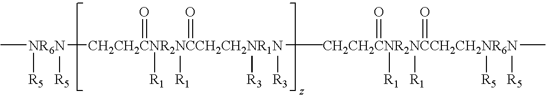

or (e) any of (a), (b), (c) or (d) wherein

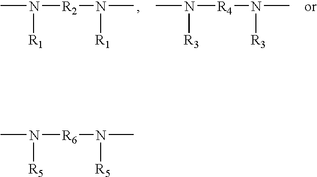

are replaced by

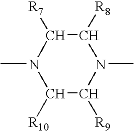

and, in any case, z is from 0 or 1 to 70 and each R₁ is independently H or a linear or branched hydrocarbon chain —CₙH₂ₙ₊₁ with n=1-4 whenever it occurs; each R₂ is independently a linear or branched alkylene chain —CₙH₂ₙ— with n=1-4 whenever it occurs; each R₃ is independently a linear or branched hydrocarbon chain —CₙH₂ₙ₊₁ with n=1-4 whenever it occurs; each R₄ is independently a linear or branched alkylene chain —CₙH₂ₙ— with n=2-4 whenever it occurs; each R₅ is independently a linear or branched hydrocarbon chain —CₙH₂ₙ₊₁ with n=1-4; each R₆ is independently a linear or branched alkylene chain —CₙH₂₉— with n=2-4; and wherein R₇, R₈, R₉ and R₁₀ are independently H or a linear or branched hydrocarbon chain —CₙH₂ₙ₊₁ with n=1-3 whenever they occur.

Advantageously, the linear polymer has the formula:

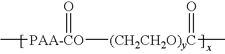

wherein PAA is a poly(amidoamine), x is from 1 to 50 and y is from 1 to 200.

Alternatively, the linear polymer has the formula

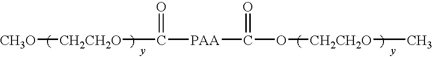

wherein PAA is a poly(amidoamine) and y is from 1 to 200.

Preferably, PAA is a poly(amidoamine) as defined above.

Advantageously, the linear polymer comprises methylbisacrylamind-dimethyethylethylenediamine.

In some preferred embodiments, the material is a liquid.

In this specification, a “naturally occurring” nucleic acid sequence is a sequence which is found in any source in nature. It is not indicative of the origin of the nucleic acid molecule, itself, (which may, for example, have been produced through artificial means) but indicates that the origin of the sequence is natural.

In order that the present invention may be more readily understood and so that further features thereof may be appreciated, embodiments of the invention will now be described, by way of example, with reference to the accompanying figures in which:

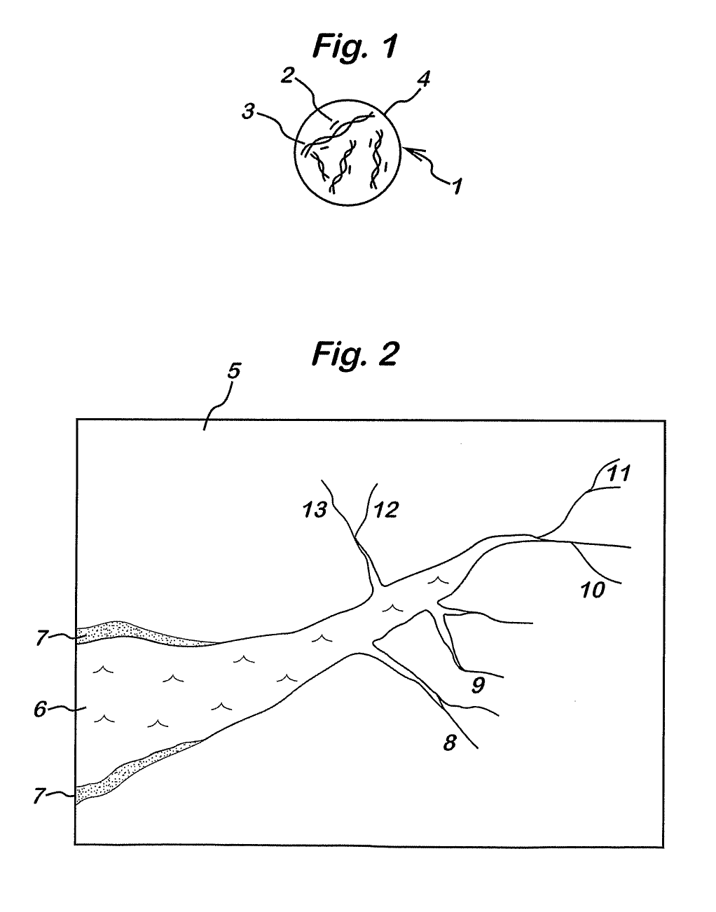

FIG. 1 is a schematic representation of a bead in accordance with one embodiment of the present invention; and

FIG. 2 is a schematic plan view of a coastal area in accordance with the method of another embodiment of the present invention.

Referring to FIG. 1, a particle or bead 1 comprises a combination of a nucleic acid tag 2, a carrier nucleic acid 3 and a linear polymer 4. The nucleic acid tag 1 comprises a plurality of identical DNA oligonucleotides of between 80 and 100 bp.

The oligonucleotides have a sequence which is selected to be unique for the tag and does not, therefore, correspond to any naturally occurring or synthetic protein-encoding sequence.

Indeed, in preferred embodiments, the nucleic acid tag comprises a stop codon within it so that, even if the sequence should become incorporated into a living organism, it cannot be expressed as a protein. In particularly preferred embodiments, three stop codons are provided, staggered into the three separate reading frames. In these embodiments, the sequence cannot be translated into a protein, irrespective of the reading frame in which it is incorporated into an organism.

In alternative embodiments, the nucleic acid tag comprises a naturally occurring sequence such as a DNA sequence of a common agricultural crop (e.g. Zea mays). Another exemplary source is salmon testes DNA. It is preferred that the tag comprises non-coding or “junk” DNA from the natural source. The advantage of using such naturally occurring DNA sequences is that there is no risk of contamination of the environment with artificial or genetically modified DNA sequences.

It is to be appreciated that, in practice, a range of different nucleic acid tags are required for marking different materials or different locations. All of the nucleic acid tags in one particular set of beads have the same identifying sequence but different sets of beads have nucleic acid tags with different sequences.

The linear polymer 4 has a backbone comprising an amido and tertiary amino groups arranged regularly on the backbone. Such polymers are disclosed in U.S. Pat. No. 6,413,941 and WO97/25067 which are each hereby incorporated by reference in their entirety. U.S. Pat. No. 6,413,941 discloses a range of examples of such polymers and provides guidance as to how nucleic acid is incorporated into the polymers. The polymer and the nucleic acid (tag plus carrier nucleic acid) are present in a ratio of, for example, 1:1. Any of the polymers disclosed in U.S. Pat. No. 6,413,941 are suitable for combination with a nucleic acid and therefore may be used in the present invention. The beads formed in the process disclosed in U.S. Pat. No. 6,413,941 are uniformly of size 130-200 nm in diameter.

The carrier nucleic add 3 comprises oligonucleotides having a different sequence from the nucleic acid tag. The exact sequence of the carrier nucleic acid is unimportant and its purpose is merely to catalyse the reaction of the formation of the linear polymer into a bead. This reaction requires a relatively large amount of nucleic add and it is more cost-effective for the bulk of the nucleic acid to comprise easily available carrier nucleic acid rather than the especially designed, and therefore expensive, nucleic acid tag. Consequently, the bead comprises at least ten times more carrier nucleic acid than nucleic acid tag but in some other embodiments, there may be 100, 1000 or 10000 times more carrier nucleic acid than nucleic acid tag. In some embodiments, the carrier nucleic acid has a naturally occurring sequence as described above in relation to the nucleic acid tag. Thus in certain embodiments, the carrier nucleic acid and the nucleic acid tag both have naturally occurring (albeit different) sequences. One preferred source of the carrier nucleic acid is salmon testes DNA, because it is available inexpensively in large amounts.

Referring to FIG. 2, the beads of one embodiment of the present invention will now be described in use. FIG. 2 is a schematic plan view of a coastal area comprising land 5, sea/estuary 6 and beach 7. Six different sets of beads are provided, the beads in each set having a unique identifying region in the nucleic acid tag sequence. In order to determine the source of the pollution of a beach 7 by animal excrement, six potential sources 8-13 of the excrement are marked, each with one of the sets of beads.

The beads are then permitted time to move unchecked, together with the material to which they have been applied, in the natural course of events. After a period of time, samples of water are taken from the beach 7 and are analysed for the presence of each set of beads. In order to conduct the analysis, the samples of water are first subjected to ultra filtration to remove matter of less than 100K Da in size and so as to increase the concentration of beads in the sample. The nucleic acid tags are extracted from the beads using guanidine hydrochloride and ethanol extraction.

The nucleic acid tags are then amplified in number, for example, by PCR. In preferred embodiments, the nucleic acid tags are amplified using “real time” PCR by the addition of fluorescently labelled DNA probes which are complementary to the identifying regions of the nucleic acid tags. The DNA probes comprise fluorophore groups, which are capable of emitting light in response to incident ultraviolet light and also comprise a quencher group which quenches the fluorescent activity when the probe is in solution. As the target nucleic acid tag is amplified, the probes hybridise to the nucleic acid tag, separating the fluorophore group from the quencher group and thus resulting in increased emission of light by the fluorophore groups. Six different types of DNA probe are provided to the PCR solution, each DNA probe being complementary to one of the nucleic tags' identifying sequence. Each of the six different types of DNA probe comprises a different fluorophore group so it is possible to measure the relative amounts of each nucleic acid tag in a single PCR reaction. The number of rounds of PCR which are required for fluorescence to reach a threshold intensity is indicative of the quantity of the respective nucleic acid tag in the sample. Further details of real time quantitation of nucleic acid tags is provided in WO00/61799 which is hereby incorporated by reference.

By determining the quantity of nucleic acid tag in the sample, it is possible to determine the concentration of each marked material on the beach 7 and, more importantly, to determine the relative importance of each potential source 8-13 in causing pollution of the beach 7. For example, if the beach 7 is sampled and 80% of the nucleic acid tag found in the sample is from one particular potential source 10 then it is indicative that the cleaning up of that potential source will result in a very significant improvement in the water quality at beach 7, even if no action is taken at the other sources 8, 9 and 11 to 13.

It is to be appreciated that the use of nucleic acid tags is particularly advantageous in such situations because a practically limitless number of different nucleic acid tags are available, owing to the variability of nucleic acid sequences and therefore a very large number of different sets of beads may be applied in a single study without risk of confusion between sets of beads. Furthermore, if a study must be repeated no delay is required in repeating the study since nucleic acid tags having different sequences can be provided in the repeat study without any risk of confusion with the tags from the first study.

### EXAMPLE

The DNA carrier and oligonucleotide tracer solution is prepared by adding highly fragmented salmon sperm DNA (Sigma-Aldrich, cat # D1626) and the single stranded oligonucleotide (CMAmplicon) tracer molecule together to give a molar ratio between 1000:1 and 10000:1 carrier DNA to oligonucleotide tracer respectively. The CMAmplicon oligonucleotide has the following sequence (5′ to 3′): GGA GAA AGA GAT GAG CTC TAA GAA AAT AAT TGG TGC TTT TGT TCT GAT GAC TGG CAT TCT GTC TGG TCA GGT ATA TGC TGG TGT AA (SEQ ID NO: 1).

The carrier DNA and oligonucleotide solutions are both prepared in phosphate buffered saline (PBS) (0.01 M phosphate buffer, 0.0027 M potassium chloride and 0.137 M sodium chloride, pH 7.4, at 25° C.) and the final DNA concentration is 0.68 g per litre of PBS).

The tracer particle solution is prepared by adding to a solution of the polymer methylbisacrylamide-dimethylethylenediamine (MBAADMEDA) in phosphate buffered saline (7.1 g polymer per litre of PBS), the solution of DNA carrier and oligonucleotides tracer. The resultant solution is gently mixed at room temperature overnight. The concentrations of both solutions are adjusted prior to mixing to ensure that the molar ratio of polymer to carrier DNA is 1:1. The polymer and DNA solutions are both made up in phosphate buffered saline (0.01 M phosphate buffer, 0.0027 M potassium chloride and 0.137 M sodium chloride, pH 7.4, at 25° C.)

The efficiency of particle formation is checked by employing photo correlation spectroscopy, using a Malvern instruments Limited Mastersizer 2000 to measure the diameter of the particles.

The efficiency of oligonucleotide incorporation into the particle solution is determined by adding 0.5 ml of the solution to a Micron YM-100 centrifugal filter device (Millipore) and spinning at 500×g for 12 minutes in a GenFuge 24D (Progen) bench top microcentrifuge. The filtrate is discarded and the retentate is made up to 0.5 ml with PBS. The Micron YM-100 is spun again at 500×g for 12 minutes, the filtrate discarded and the retentate made up to 0.5 ml with PBS. DNA is extracted from 200 ul of the retentate using the QIAmp DNA Mini Kit (Qiagen™) and following the procedure listed in the accompanying handbook (see the TissueProtocol, page 34, step 3a et seq from the QIAamp DNA Mini Kit and QIAamp DNA Bland Mini KA 02/2003). The oligonucleotide tracer concentration is the final Qiagen protocol extract is quantified by undertaking a qPCR reaction using the following primers, probe and reaction conditions:

Forward primer (5′-3′) (SEQ ID NO: 2):
GGA GAA AGA GAT GAG CTC TAA GAA AAT AA
Reverse primer (5′-3′) (SEQ ID NO: 3):
TTA CAC CAG CAT ATA CCT GAC CAG A
Taqman type probe (5′-3′) (SEQ ID NO: 4):
TGG TGC TTT TGT TCT GAT GAC TGG CAT TCT

(FAM labelled at 5′ end, TAMRA labelled at 3′ end)

The primers are used at a final concentration of 900 nM, the probe is used at a final concentration of 125 nM. The qPCR reaction is made up in 2× TaqMan Universal PCR Master Mix (Applied Blosystems). The final reaction volume comprises 22.5 μl of the 1× Master Mix with primers and probe and 2.5 μl of the Qiagen final DNA extract. The qPCR assay is undertaken using an Applied Biosystems 7300 Real Time PCR System operated with the following thermal cycler conditions: 2 minutes at 50° C. and 10 minutes at 95° C. followed by 40 cycles of 95° C. for 15 seconds and 60° C. for 1 minute. The concentration of the tracer oligonucleotide is calculated by comparing the Ct value obtained against a standard curve generated using 10-fold dilutions of the CMAmplicon run under identical conditions.

The efficiency of tracer oligonucleotide incorporation into the tracer particles is then calculated by back calculation.

The original particle solution is then amended with bovine serum albumin (Sigma-Aldrich, cat # A3902) to give a final concentration of 0.05% (w/w). The tracer solution has a nominal particle concentration of 1×10¹² particles per ml.

The tracer is transported to the study area and is added to the watercourse through the use of a metered controllable pump to give a final concentration in the watercourse at the point of addition of 1×10¹² particles per cubic meter of water tagged.

1 litre samples are collected at the various sampling points and are transported to the laboratory where they are processed as follows. 250 ml of the sample supernatant is centrifuged at 4000 rpm in a Sigma bench top centrifuge to remove particulate matter. 200 ml of the clarified supernatant is added to an Amicon 200 ml Stirred Cell 8200 (Millipore cat #5123) equipped with a 100K Dalton ultrafiltration membrane. The sample is reduced in volume until approx. 5 ml of retentate is left. The retentate is recovered and its volume measured. 3 ml of the retentate is then added to an Amicon 3 ml Stirred Cell 8003 (Millipore cat #5125) equipped with a 100K Dalton ultrafiltration membrane and the volume reduced to approx. 250 ul. The retentate is recovered and its volume measured.

The presence and concentration of the oligonucleotide tracer in the Amicon 3 ml stirred cell retentate is determined by extracting 200 ul of the retentate using the QIAmp DNA Mini Kit (Qiagen) and undertaking qPCR reactions on the Qiagen extract as described above.

1-30. (canceled)

31. A method of marking a material comprising the steps of:
i) providing one or more marker particles; and
ii) applying the marker particles to the material, wherein the marker particles comprise a linear polymer with a backbone comprising amido and tertiary amino groups arranged regularly on the backbone and a nucleic acid tag.

32. A method of detecting whether a material has been marked in accordance with claim 1 comprising the steps of:
i) sampling a portion of the material; and
ii) detecting the presence of the nucleic acid tag in the sample.

33. A method according to claim 32 wherein step ii) further comprises the step of concentrating the amount of nucleic acid tag in the sample by filtration.

34. A method according to claim 32 wherein step ii) further comprises the step of extracting the nucleic acid tag from the marker particles.

35. A method according to claim 32 wherein step ii) further comprises the step of determining the quantity of nucleic acid tag present in the sample.

36. A method according to claim 35 wherein the quantity of nucleic acid tag present in the sample is determined by real time PCR.

37. A method of marking a plurality of materials comprising the steps of claim 31 wherein each material is marked with a separate set of marker particles, the marker particles in each set comprising nucleic acid tag having a different sequence.

38. A method according to claim 37 wherein the nucleic acid tags in each set of marker particles comprise a different identifying sequence flanked by generic sequences shared by the nucleic acid tags in all sets.

39. The method of claim 31 wherein the linear polymer comprises or consists of a poly(amidoamine).

40. The method according to claim 31 wherein the linear polymer further comprises additional substituents selected from the group consisting of ethylene glycol and poly(ethylene glycol).

41. The method according to claim 40 wherein said linear polymer is selected form the group consisting of a poly(ethylene glycol)-poly(amidoamine) block copolymer and a ethylene glycol-poly(amidoamine) block copolymer.

42. The method according to claim 41 wherein said linear polymer is a block copolymer with the structure
[poly(amidoamine)-(ethylene glycol)_{y}]ₓ
wherein x is from 1 to 50 and each y is independently from 1 to 200 whenever it occurs.

43. The method according to claim 41 wherein said linear polymer is a block copolymer with the structure
(ethylene glycol)_{y}-poly(amidoamine)-(ethylene glycol)_{y}
wherein each y is independently 1 to 200.

44. The method according to claim 39 wherein said poly(amidoamine) has the formula selected from the group consisting of:
(a) 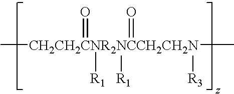
and (b) 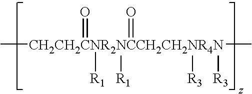
and (c) 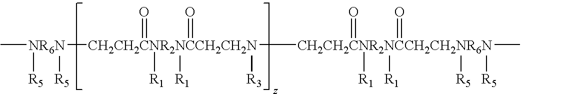
and (d) 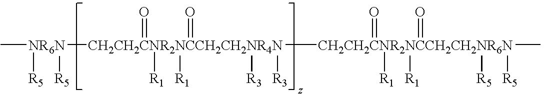
and (e) any of (a), (b), (c) or (d) wherein 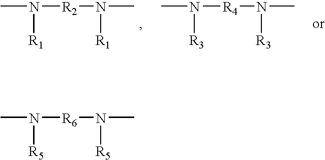
are replaced by 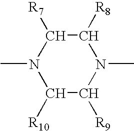
and, in any case, z is from 0 or 1 to 70 and each R₁ is independently H or a linear or branched hydrocarbon chain —CₙH₂ₙ₊₁ with n=1-4 whenever it occurs; each R₂ is independently a linear or branched alkylene chain —CₙH₂ₙ— with n=1-4 whenever it occurs; each R₃ is independently a linear or branched hydrocarbon chain —CₙH₂ₙ₊₁ with n=1-4 whenever it occurs; each R₄ is independently a linear or branched alkylene chain —CₙH₂ₙ— with n=2-4 whenever it occurs; each R₅ is independently a linear or branched hydrocarbon chain —CₙH₂ₙ₊₁ with n=1-4; each R₆ is independently a linear or branched alkylene chain —CₙH₂ₙ— with n=2-4; and wherein R₇, R₈, R₉ and R₁₀ are independently H or a linear or branched hydrocarbon chain —CₙH₂ₙ₊₁ with n=1-3 whenever they occur.

45. The method according to claim 40 wherein the linear polymer has the formula: 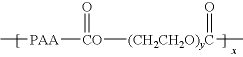
wherein PAA is a poly(amidoamine), x is from 1 to 50 and y is from 1 to 200.

46. The method according to claim 40 wherein the linear polymer has the formula 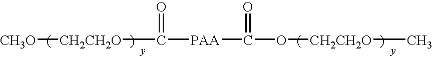
wherein PAA is a poly(amidoamine) and y is from 1 to 200.

47. The method according to claim 45 wherein PAA is a poly(amidoamine) having the formula selected from the group consisting of:
(a) 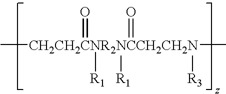
and (b) 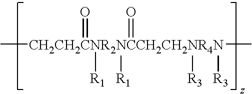
and (c) 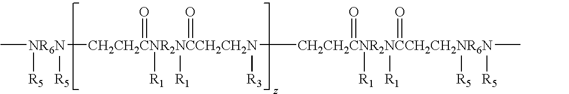
and (d) 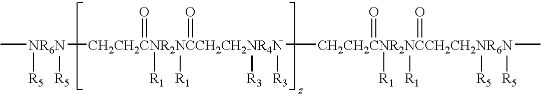
and (e) any of (a), (b), (c) or (d) wherein 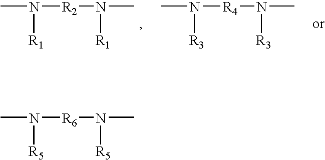
are replaced by 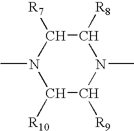
and, in any case, z is from 0 or 1 to 70 and each R₁ is independently H or a linear or branched hydrocarbon chain —CₙH₂ₙ₊₁ with n=1-4 whenever it occurs; each R₂ is independently a linear or branched alkylene chain —CₙH₂ₙ— with n=1-4 whenever it occurs; each R₃ is independently a linear or branched hydrocarbon chain —CₙH₂ₙ₊₁ with n=1-4 whenever it occurs; each R₄ is independently a linear or branched alkylene chain —CₙH₂ₙ— with n=2-4 whenever it occurs; each R₅ is independently a linear or branched hydrocarbon chain —CₙH₂ₙ₊₁ with n=1-4; each R₆ is independently a linear or branched alkylene chain —CₙH₂ₙ— with n=2-4; and wherein R₇, R₈, R₉ and R₁₀ are independently H or a linear or branched hydrocarbon chain —CₙH₂ₙ₊₁ with n=1-3 whenever they occur.

48. The method according to claim 46 wherein PAA is a poly(amidoamine) having the formula selected from the group consisting of:
(a) 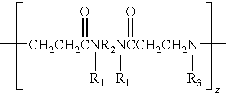
and (b) 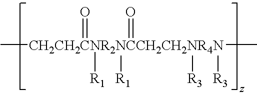
and (c) 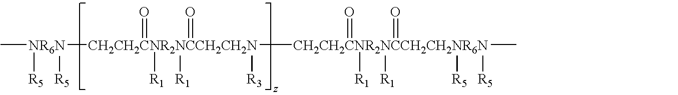
and (d) 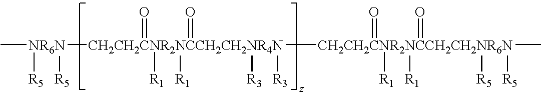
and (e) any of (a), (b), (c) or (d) wherein 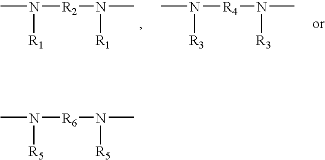
are replaced by 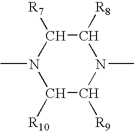
and, in any case, z is from 0 or 1 to 70 and each R₁ is independently H or a linear or branched hydrocarbon chain —CₙH₂ₙ₊₁ with n=1-4 whenever it occurs; each R₂ is independently a linear or branched alkylene chain —CₙH₂ₙ— with n=1-4 whenever it occurs; each R₃ is independently a linear or branched hydrocarbon chain —CₙH₂ₙ₊₁ with n=1-4 whenever it occurs; each R₄ is independently a linear or branched alkylene chain —CₙH₂ₙ— with n=2-4 whenever it occurs; each R₅ is independently a linear or branched hydrocarbon chain —CₙH₂ₙ₊₁ with n=1-4; each R₆ is independently a linear or branched alkylene chain —CₙH₂ₙ— with n=2-4; and wherein R₇, R₈, R₉ and R₁₀ are independently H or a linear or branched hydrocarbon chain —CₙH₂ₙ₊₁ with n=1-3 whenever they occur.

49. The method according to claim 39 wherein the linear polymer comprises methylbisacrylamind-dimethylethylethylenediamine.

50. The use of a composition comprising a linear polymer with a backbone comprising amido and tertiary amino groups arranged regularly on the backbone and a nucleic acid tag, for marking a material.

51. The use of claim 50 wherein the linear polymer comprises or consists of a poly(amidoamine).

52. The use according to claim 50 wherein the linear polymer further comprises additional substituents selected from the group consisting of ethylene glycol and poly(ethylene glycol).

53. The use according to claim 52 wherein said linear polymer is selected form the group consisting of a poly(ethylene glycol)-poly(amidoamine) block copolymer and a ethylene glycol-poly(amidoamine) block copolymer.

54. The use according to claim 53 wherein said linear polymer is a block copolymer with the structure
[poly(amidoamine)-(ethylene glycol)_{y}]ₓ
wherein x is from 1 to 50 and each y is independently from 1 to 200 whenever it occurs.

55. The use according to claim 53 wherein said linear polymer is a block copolymer with the structure
(ethylene glycol)_{y}-poly(amidoamine)-(ethylene glycol)_{y}
wherein each y is independently 1 to 200.

56. The use according to claim 51 wherein said poly(amidoamine) has the formula selected from the group consisting of:
(a) 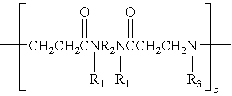
and (b) 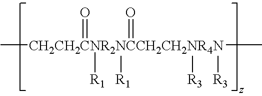
and (c) 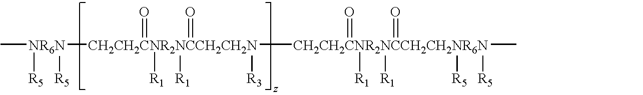
and (d) 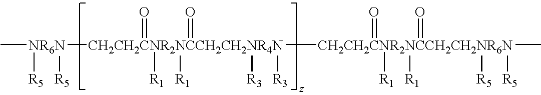
and (e) any of (a), (b), (c) or (d) wherein 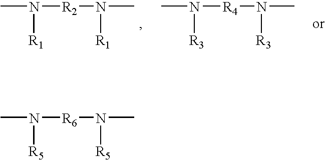
are replaced by 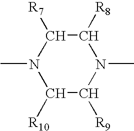
and, in any case, z is from 0 or 1 to 70 and each R₁ is independently H or a linear or branched hydrocarbon chain —CₙH₂ₙ₊₁ with n=1-4 whenever it occurs; each R₂ is independently a linear or branched alkylene chain —CₙH₂ₙ— with n=1-4 whenever it occurs; each R₃ is independently a linear or branched hydrocarbon chain —CₙH₂ₙ₊₁ with n=1-4 whenever it occurs; each R₄ is independently a linear or branched alkylene chain —CₙH₂ₙ— with n=2-4 whenever it occurs; each R₅ is independently a linear or branched hydrocarbon chain —CₙH₂ₙ₊₁ with n=1-4; each R₆ is independently a linear or branched alkylene chain —CₙH₂ₙ— with n=2-4; and wherein R₇, R₈, R₉ and R₁₀ are independently H or a linear or branched hydrocarbon chain —CₙH₂ₙ₊₁ with n=1-3 whenever they occur.

57. The use according to claim 52 wherein the linear polymer has the formula: 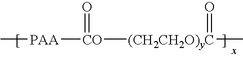
wherein PAA is a poly(amidoamine), x is from 1 to 50 and y is from 1 to 200.

58. The use according to claim 52 wherein the linear polymer has the formula 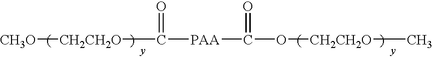
wherein PAA is a poly(amidoamine) and y is from 1 to 200.

59. The use according to claim 57 wherein PAA is a poly(amidoamine) having the formula selected from the group consisting of:
(a) 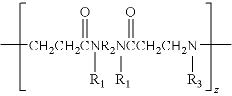
and (b) 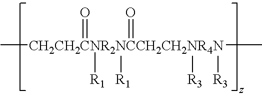
and (c) 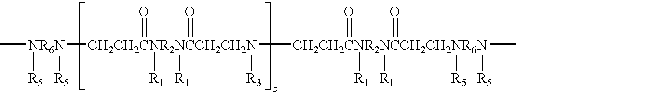
and (d) 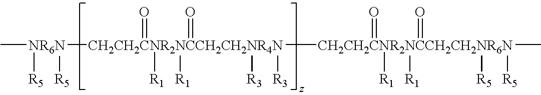
and (e) any of (a), (b), (c) or (d) wherein 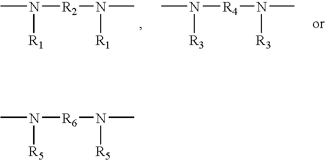
are replaced by 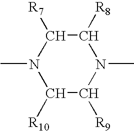
and, in any case, z is from 0 or 1 to 70 and each R₁ is independently H or a linear or branched hydrocarbon chain —CₙH₂ₙ₊₁ with n=1-4 whenever it occurs; each R₂ is independently a linear or branched alkylene chain —CₙH₂ₙ— with n=1-4 whenever it occurs; each R₃ is independently a linear or branched hydrocarbon chain —CₙH₂ₙ₊₁ with n=1-4 whenever it occurs; each R₄ is independently a linear or branched alkylene chain —CₙH₂ₙ— with n=2-4 whenever it occurs; each R₅ is independently a linear or branched hydrocarbon chain —CₙH₂ₙ₊₁ with n=1-4; each R₆ is independently a linear or branched alkylene chain —CₙH₂ₙ— with n=2-4; and wherein R₇, R₈, R₉ and R₁₀ are independently H or a linear or branched hydrocarbon chain —CₙH₂ₙ₊₁ with n=1-3 whenever they occur.

60. The use according to claim 58 wherein PAA is a poly(amidoamine) having the formula selected from the group consisting of:
(a) 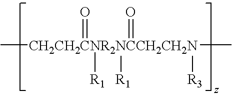
and (b) 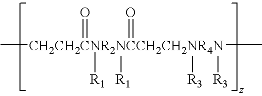
and (c) 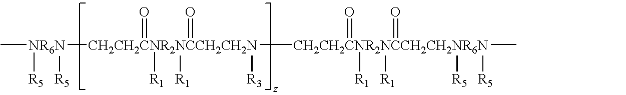
and (d) 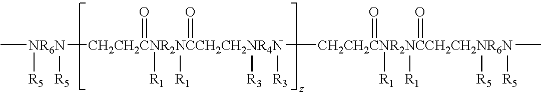
and (e) any of (a), (b), (c) or (d) wherein 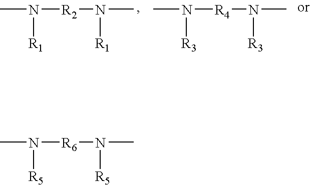
are replaced by 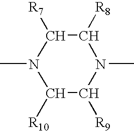
and, in any case, z is from 0 or 1 to 70 and each R₁ is independently H or a linear or branched hydrocarbon chain —CₙH₂ₙ₊₁ with n=1-4 whenever it occurs; each R₂ is independently a linear or branched alkylene chain —CₙH₂ₙ— with n=1-4 whenever it occurs; each R₃ is independently a linear or branched hydrocarbon chain —CₙH₂ₙ₊₁ with n=1-4 whenever it occurs; each R₄ is independently a linear or branched alkylene chain —CₙH₂ₙ— with n=2-4 whenever it occurs; each R₅ is independently a linear or branched hydrocarbon chain —CₙH₂ₙ₊₁ with n=1-4; each R₆ is independently a linear or branched alkylene chain —CₙH₂ₙ— with n=2-4; and wherein R₇, R₈, R₉ and R₁₀ are independently H or a linear or branched hydrocarbon chain —CₙH₂ₙ₊₁ with n=1-3 whenever they occur.

61. The use according to claim 51 wherein the linear polymer comprises methylbisacrylam.


SEQUENCE LISTING (4 sequences)

SEQ ID NO: 1 - DNA, 86 bp, Artificial Sequence
  Single stranded oligonucleotide tracer molecule
ggagaaagagatgagctctaagaaaataattggtgcttttgttctgatgactggcattctgtctggtcaggtatatgctggtgtaa

SEQ ID NO: 2 - DNA, 29 bp, Artificial Sequence
  Forward primer for SEQ ID NO 1
ggagaaagagatgagctctaagaaaataa

SEQ ID NO: 3 - DNA, 25 bp, Artificial Sequence
  Reverse primer for SEQ ID NO 1
ttacaccagcatatacctgaccaga

SEQ ID NO: 4 - DNA, 30 bp, Artificial Sequence
  Taqman type probe for SEQ ID NO 1
tggtgcttttgttctgatgactggcattct